(19) United States
(12) Patent Application Publication
(10) Pub. No.: US 2004/0142007 A1
(43) Pub. Date: Jul. 22, 2004

(54) Cosmetic preparations containing an extract of germinating plants

(76) Inventors: Philippe Moussou, Nancy (FR); Louis Danoux, Nancy (FR); Bruno Daridon, Malzeville (FR); Gilles Pauly, Nancy (FR)

Correspondence Address: COGNIS CORPORATION, PATENT DEPARTMENT, 300 BROOKSIDE AVENUE, AMBLER, PA, 19002, US

(21) Appl. No.: 10/479,336

(22) PCT Filed: May 23, 2002
(86) PCT No.: PCT/EP02/05660

(30) Foreign Application Priority Data
Jun. 1, 2001 (EP) 01401428.6

(51) Int. Cl. A61K 6/00

(52) U.S. Cl. 424/401

(57) ABSTRACT

The invention relates to novel cosmetic preparations containing an effective amount of an extract of germinating plants.

### FIELD OF THE INVENTION

This invention relates generally to cosmetic products and, more particularly, to new preparations with an effective content of extracts of germinating plants.

### PRIOR ART

Modern cosmetic preparations are having to meet increasingly more stringent consumer requirements. Long gone are the days when it was sufficient for a night cream to give moisture back to the skin. Today, the preparation is also expected to protect against environmental stress and to repair damage. Apart from the fact that such requirements presuppose a deep understanding of the biochemical processes in the skin and hair which, even today, is still not completely present, many different active components which take account of the many triggering factors are also generally needed to meet this requirement. Apart from the possibly unwanted interaction of such artificially composed mixtures, there is of course also the problem of storage and the technical difficulties involved in production which make such complex products difficult to develop and expensive. On the other hand, it will readily be appreciated why the cosmetics industry has a considerable interest in active components, especially vegetable active components, which have a “broadband effect”, i.e. solve different cosmetic problems at one and the same time. Particular interest naturally attaches to natural processes in plants in the course of which numerous substances are formed alongside one another and complement one another in their activity.

Germination is the umbrella term for very different biochemical processes such as, for example, protein hydration, subcellular structural changes, respiration, synthesis of macromolecules and the general expansion of the cells. The common goal of all these processes is to convert the dehydrated plant embryo present in the resting seed into a germinating sprout. This requires certain environmental conditions, for example a suitable temperature and the presence of oxygen. The process of germination involves a large number of substances, more particularly growth factors, such as, for example, auxins, gibberellins and cytokinins, and enzymes which mobilize storage substances, such as, for example, carbohydrates, proteins, triacylglycerol or phytin. The organism itself produces endogenous enzymes (amylases, pentosanases, glucanases, proteinases, lipases, phytases, etc.), in order to metabolize the macromolecules so that the smaller fragments can be transported to the place where they are needed.

In this connection, reference is made, for example, to an article by Malak [Cosmetol. 20, 44 (1998)] which describes the use of a malt extract as an inhibitor for matrix metalloproteinases, such as collagenase for example. In Ärztl. Kosmetol. 17, 342-352 (1987), Tronnier also reports on its anti-inflammatory activity. The immunostimulating activity of extracts of germinating plants is known from DE 4141866 A1. In NCP, 224, 4-7 (1997), Benaiges et al. report on the anti-elastase activity of extracts of germinating alfalfa sprouts. WO 99/56712 (Provital) relates to the use of extracts of germinating plants for stimulating cell respiration. FR 2665900 A1 (Andromaco) describes the use of oligosaccharides obtained from germinating plants for healing wounds, disclosing the stimulation of the lymphoblasts which, however, are neither skin nor hair cells. Finally, FR 2747044 A1 (Coletica) describes the use of superoxide dismutase obtained from germinating plants in cosmetic preparations.

Accordingly, the problem addressed by the present invention was to provide new cosmetic active components using vegetable active substances which would solve various problems at one and the same time. In particular, the new cosmetic active components would counteract ageing of the skin and slackness of the connective tissue (cellulite), simulate the synthesis of lipids in the stratum corneum and thus strengthen the skin barrier, support skin and hair follicles in providing protection against environmental poisons, oxidative stress and UV radiation, promote the synthesis of macromolecules, such as collagen for example, in the fibroblasts, prevent the inflammation of sensitive human skin and support the lipolysis and the purification of body cells.

### DESCRIPTION OF THE INVENTION

The present invention relates to cosmetic preparations containing an effective quantity of an extract of germinating plants.

It has surprisingly been found that the extracts have the desired broad spectrum of effects and may therefore be used for the care and protection of the skin and hair.

Plant Extracts

Within certain limits, germinating plants always contain the same active substances with which the described broad cosmetic effect can be obtained in accordance with the invention. Accordingly, their choice is not critical. Typical examples are alfalfa, bambara nuts, carob, borage, broccoli, buck wheat, Chinese cabbage, peas, peanuts, flax, fennel, cloves, carrots, cress, lentils, corn, melon, parsley, rape, radishes, rice, red cabbage, celery, mustard, sesame, soya, sunflowers, onions and cereals, such as rye, wheat, Kamut® wheat, barley, oats and spelt. Mixtures may of course also be used. The effective quantity of the extracts is generally 0.01 to 5, preferably 0.1 to 2 and more particularly 0.05 to 1% by weight, based of course on the active substance content of the extracts and the percentage content of the extracts in the final formulation.

Extraction

The extracts may be prepared by methods known per se, i.e. for example by aqueous, alcoholic or aqueous/alcoholic extraction of the plants or parts thereof. Particulars of suitable conventional extraction processes, such as maceration, remaceration, digestion, agitation maceration, vortex extraction, ultrasonic extraction, countercurrent extraction, percolation, repercolation, evacolation (extraction under reduced pressure), diacolation and solid/liquid extraction under continuous reflux in a Soxhlet extractor, which are familiar to the expert and which may all be used in principle, can be found, for example, in Hagers Handbuch der pharmazeutischen Praxis (5th Edition, Vol. 2, pp. 1026-1030, Springer Verlag, Berlin-Heidelberg-New York 1991). Percolation is advantageous for industrial use. Fresh plants or parts thereof are suitable as the starting material although dried plants and/or plant parts which may be mechanically size-reduced before extraction are normally used. Any size reduction methods known to the expert, for example freeze grinding, may be used. Preferred solvents for the extraction process are organic solvents, water (preferably hot water with a temperature above 80° C. and more particularly above 95° C.) or mixtures of organic solvents and water, more particularly low molecular weight alcohols with more or less high water contents. Extraction with methanol, ethanol, pentane, hexane, heptane, acetone, propylene glycols, polyethylene glycols, ethyl acetate and mixtures and water-containing mixtures thereof is particularly preferred. The extraction process is generally carried out at 20 to 100° C., preferably at 30 to 90° C. and more particularly at 60 to 80° C. In one preferred embodiment, the extraction process is carried out in an inert gas atmosphere to avoid oxidation of the ingredients of the extract. This is particularly important where extraction is carried out at temperatures above 40° C. The extraction times are selected by the expert in dependence upon the starting material, the extraction process, the extraction temperature and the ratio of solvent to raw material, etc. After the extraction process, the crude extracts obtained may optionally be subjected to other typical steps, such as for example purification, concentration and/or decoloration. If desired, the extracts thus prepared may be subjected, for example, to the selective removal of individual unwanted ingredients. The extraction process may be carried out to any degree, but is usually continued to exhaustion. Typical yields (=extract dry matter, based on the quantity of raw material used) in the extraction of dried leaves are in the range from 3 to 15 and more particularly 6 to 10% by weight. The present invention includes the observation that the extraction conditions and the yields of the final extracts may be selected according to the desired application. These extracts, which generally have active substance contents (=solids contents) of 0.5 to 10% by weight, may be used as such, although the solvent may also be completely removed by drying, more particularly by spray or freeze drying, a deep red colored solid remaining behind. The extracts may also be used as starting materials for producing the pure active substances mentioned above unless they can be synthesized by a more simple and inexpensive method. Accordingly, the active substance content in the extracts may be from 5 to 100% by weight and is preferably from 50 to 95% by weight. The extracts themselves may be present as water-containing preparations and/or as preparations dissolved in organic solvents and as spray-dried or freeze-dried water-free solids. Suitable organic solvents in this connection are, for example, aliphatic alcohols containing 1 to 6 carbon atoms (for example ethanol), ketones (for example acetone), halogenated hydrocarbons (for example chloroform or methylene chloride), lower esters or polyols (for example glycerol or glycols).

In a preferred embodiment of the invention, however, the extracts are produced by first extracting the germinating seeds with water and/or alcohol and then heat-treating them in a first step in which the temperature is slowly increased to 100° C. to activate the enzymes needed to metabolize the storage substances. After a certain time, the temperature is increased beyond 100° C. to deactivate the enzymes. This is followed by the optional addition of exogenases which are intended to terminate the hydrolysis started by the endogenases already present in the extracts. The extracts may then be dried as described above.

Commercial Applications

As explained above, a focal point of the invention is generally the use of extracts of germinating plants for the production of cosmetic preparations in which they may be present in quantities of 0.01 to 25, preferably 0.1 to 15 and more particularly 1 to 5% by weight. The present invention also relates to the use of the extracts

for stimulating cell growth and the cell metabolism;

for stimulating the renewal of dermal macromolecules by the fibroblasts;

for stimulating cell protein synthesis for protection against spontaneous ageing effects;

for increasing the protein and GSH concentrations in the cells (strengthening the defence against environmental influences);

for stimulating G6PDH activity against dry and rough skin;

for immunomodulation;

as anti-inflammatory agents;

as active components against acne and rosacea;

as antioxidants;

for protecting the skin and hair against the effects of UVA and UVB radiation;

for protecting sensitive skin;

an anti-stress agents;

for stimulating the synthesis and release of heat shock proteins;

as lipolytic agents;

as active components for purifying body cells;

as active components for inhibiting the synthesis of melanin in skin and hair cells;

as active components with oestrogen-like activity.

Cosmetic Preparations

The cosmetic preparations and especially the skin treatment preparations for sensitive skin may contain mild surfactants, oil components, emulsifiers, pearlizing waxes, consistency factors, thickeners, superfatting agents, stabilizers, polymers, silicone compounds, fats, waxes, lecithins, phospholipids, biogenic agents, UV protection factors, antioxidants, deodorants, antiperspirants, film formers, swelling agents, insect repellents, self-tanning agents, tyrosine inhibitors (depigmenting agents), hydrotropes, solubilizers, perservatives, perfume oils, dyes and the like as further auxiliaries and additives.

Surfactants

Suitable surfactants are anionic, nonionic, cationic and/or amphoteric or zwitterionic surfactants which may be present in the preparations in quantities of normally about 1 to 70% by weight, preferably 5 to 50% by weight and more preferably 10 to 30% by weight. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, aminopropionates, aminoglycinates, imidazolinium betaines and sulfobetaines. The surfactants mentioned are all known compounds. Information on their structure and production can be found in relevant synoptic works, cf. for example J. Falbe (ed.), “Surfactants in Consumer Products”, Springer Verlag, Berlin, 1987, pages 54 to 124 or J. Falbe (ed.), “Katalysatoren, Tenside und Mineralöladditive (Catalysts, Surfactants and Mineral Oil Additives)”, Thieme Verlag, Stuttgart, 1978, pages 123-217. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines, amphoacetals and/or protein fatty acid condensates, preferably based on wheat proteins.

Oil Components

Suitable oil components are, for example, Guerbet alcohols based on fatty alcohols containing 6 to 18 and preferably 8 to 10 carbon atoms, esters of linear C₆₋₂₂ fatty acids with linear or branched C₆₋₂₂ fatty alcohols or esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₁₈₋₃₈ alkylhydroxycarboxylic acids with linear or branched C₆₋₂₂ fatty alcohols (cf. DE 197 56 377 A1), more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di- and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, such as Dicaprylyl Carbonate (Cetiol® CC) for example, Guerbet carbonates based on C₆₋₁₈ and preferably C₈₋₁₀ fatty alcohols, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group, such as Dicaprylyl Ether (Cetiol® OE) for example, ring opening products of epoxidized fatty acid esters with polyols, silicone oils (cyclomethicone, silicon methicone types, etc.) and/or aliphatic or naphthenic hydrocarbons such as, for example, squalane, squalene or dialkyl cyclohexanes.

Emulsifiers

Suitable emulsifiers are, for example, nonionic surfactants from at least one of the following groups:

products of the addition of 2 to 30 mol ethylene oxide and/or 0 to 5 mol propylene oxide onto linear C₈₋₂₂ fatty alcohols, onto C₁₂₋₂₂ fatty acids, onto alkyl phenols containing 8 to 15 carbon atoms in the alkyl group and onto alkylamines containing 8 to 22 carbon atoms in the alkyl group;

alkyl and/or alkenyl oligoglycosides containing 8 to 22 carbon atoms in the alk(en)yl group and ethoxylated analogs thereof;

addition products of 1 to 15 mol ethylene oxide onto castor oil and/or hydrogenated castor oil;

addition products of 15 to 60 mol ethylene oxide onto castor oil and/or hydrogenated castor oil;

partial esters of glycerol and/or sorbitan with unsaturated, linear or saturated, branched fatty acids containing 12 to 22 carbon atoms and/or hydroxycarboxylic acids containing 3 to 18 carbon atoms and addition products thereof onto 1 to 30 mol ethylene oxide;

partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and addition products thereof onto 1 to 30 mol ethylene oxide;

mixed esters of pentaerythritol, fatty acids, citric acid and fatty alcohol according to DE 1165574 PS and/or mixed esters of fatty acids containing 6 to 22 carbon atoms, methyl glucose and polyols, preferably glycerol or polyglycerol,

mono-, di- and trialkyl phosphates and mono-, di- and/or tri-PEG-alkyl phosphates and salts thereof,

wool wax alcohols,

polysiloxane/polyalkyl/polyether copolymers and corresponding derivatives,

block copolymers, for example Polyethyleneglycol-30 Dipolyhydroxystearate;

polymer emulsifiers, for example Pemulen types (TR-1, TR-2) of Goodrich;

polyalkylene glycols and

glycerol carbonate.

Ethylene Oxide Addition Products

The addition products of ethylene oxide and/or propylene oxide onto fatty alcohols, fatty acids, alkylphenols or onto castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of addition products of ethylene oxide onto glycerol are known as lipid layer enhancers for cosmetic formulations from DE 2024051 PS.

Alkyl and/or Alkenyl Oligoglycosides

Alkyl and/or alkenyl oligoglycosides, their production and their use are known from the prior art. They are produced in particular by reacting glucose or oligosaccharides with primary alcohols containing 8 to 18 carbon atoms. So far as the glycoside unit is concerned, both monoglycosides in which a cyclic sugar unit is attached to the fatty alcohol by a glycoside bond and oligomeric glycosides with a degree of oligomerization of preferably up to about 8 are suitable. The degree of oligomerization is a statistical mean value on which the homolog distribution typical of such technical products is based.

Partial Glycerides

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide onto the partial glycerides mentioned are also suitable.

Sorbitan Esters

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide onto the sorbitan esters mentioned are also suitable.

Polyglycerol Esters

Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylolpropane or pentaerythritol with lauric acid, cocofatty acid, tallow fatty acid, palmitic acid, stearic acid, oleic acid, behenic acid and the like optionally reacted with 1 to 30 mol ethylene oxide.

Anionic Emulsifiers

Typical anionic emulsifiers are aliphatic fatty acids containing 12 to 22 carbon atoms such as, for example, palmitic acid, stearic acid or behenic acid and dicarboxylic acids containing 12 to 22 carbon atoms such as, for example, azelaic acid or sebacic acid.

Amphoteric and Cationic Emulsifiers

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-coco-alkylaminopropionate, cocoacylaminoethyl aminopropionate and C_{12/18} acyl sarcosine. Finally, cationic surfactants are also suitable emulsifiers, those of the esterquat type, preferably methyl-quaternized difatty acid triethanolamine ester salts, being particularly preferred.

Fats and Waxes

Typical examples of fats are glycerides, i.e. solid or liquid, vegetable or animal products which consist essentially of mixed glycerol esters of higher fatty acids. Suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycerophospholipids which are formed from fatty acids, glycerol, phosphoric acid and choline by esterification. Accordingly, lecithins are also frequently referred to by experts as phosphatidyl cholines (PCs). Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycerophosphates) which are normally classed as fats. Sphingosines and sphingolipids are also suitable.

Pearlizing Waxes

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Consistency Factors and Thickeners

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used. Suitable thickeners are, for example, Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® and Pemulen types [Goodrich]; Synthalens® [Sigma]; Keltrol types [Kelco]; Sepigel types [Seppic]; Salcare types [Allied Colloids]), polyacrylamides, polymers, polyvinyl alcohol and polyvinyl pyrrolidone. Other consistency factors which have proved to be particularly effective are bentonites, for example Bentone® Gel VS-5PC (Rheox) which is a mixture of cyclopentasiloxane, Disteardimonium Hectorite and propylene carbonate. Other suitable consistency factors are surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Superfatting Agents

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Stabilizers

Metal salts of fatty acids such as, for example, magnesium, aluminium and/or zinc stearate or ricinoleate may be used as stabilizers.

Polymers

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grünau), quaternized wheat polypeptides, polyethyleneimine, cationic silicone polymers such as, for example, amodimethicone, copolymers of adipic acid and dimethylaminohydroxypropyl diethylenetriamine (Cartaretine®, Sandoz), copolymers of acrylic acid with dimethyl diallyl ammonium chloride (Merquat® 550, Chemviron), polyaminopolyamides as described, for example, in FR 2252840 A and crosslinked water-soluble polymers thereof, cationic chitin derivatives such as, for example, quaternized chitosan, optionally in micro-crystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamido-propyl trimethylammonium chloride/acrylate copolymers, octylacryl-amide/methyl methacrylate/tert.-butylaminoethyl methacrylate/2-hydroxy-propyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones. Other suitable polymers and thickeners can be found in Cosm. Toil., 108, 95 (1993).

Silicone Compounds

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates. A detailed overview of suitable volatile silicones can be found in Todd et al. in Cosm. Toil. 91, 27 (1976).

UV Protection Factors and Antioxidants

UV protection factors in the context of the invention are, for example, organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. The following are examples of oil-soluble substances:

3-benzylidene camphor or 3-benzylidene norcamphor and derivatives thereof, for example 3-(4-methylbenzylidene)-camphor as described in EP 0693471 B1;

4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester;

esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);

esters of salicylic acid, preferably salicylic acid-2-ethylhexyl ester, salicylic acid-4-isopropylbenzyl ester, salicylic acid homomenthyl ester;

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzalmalonic acid, preferably 4-methoxybenzalmalonic acid di-2-ethylhexyl ester;

triazine derivatives such as, for example, 2,4,6-trianilino-(p-carbo-2′-ethyl-1′-hexyloxy)-1,3,5-triazine and Octyl Triazone as described in EP 0818450 Alor Dioctyl Butamido Triazone (Uvasorb® HEB);

propane-1,3-diones such as, for example, 1-(4-tert.butylphenyl)-3-(4′-methoxyphenyl)-propane-1,3-dione;

ketotricyclo(5.2.1.0)decane derivatives as described in EP 0694521 B1.

Suitable water-soluble substances are

2-phenylbenzimidazole-5-sulfonic acid and alkali metal, alkaline earth metal, ammonium, alkylammonium, alkanolammonium and glucammonium salts thereof;

sulfonic acid derivatives of benzophenones, preferably 2-hydroxy-4-methoxybenzophenone-5-sulfonic acid and salts thereof;

sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol® 1789) or 1-phenyl-3-(4′-isopropylphenyl)-propane-1,3-dione and the enamine compounds described in DE 19712033 A1 (BASF). The UV-A and UV-B filters may of course also be used in the form of mixtures. Particularly favorable combinations consist of the derivatives of benzoyl methane, for example 4-tert.butyl-4′-methoxydibenzoylmethane (Parsol® 1789) and 2-cyano-3,3-phenylcinnamic acid-2-ethyl hexyl ester (Octocrylene) in combination with esters of cinnamic acid, preferably 4-methoxycinnamic acid-2-ethyl hexyl ester and/or 4-methoxycinnamic acid propyl ester and/or 4-methoxycinnamic acid isoamyl ester. Combinations such as these are advantageously combined with water-soluble filters such as, for example, 2-phenylbenzimidazole-5-sulfonic acid and alkali metal, alkaline earth metal, ammonium, alkylammonium, alkanolammonium and glucammonium salts thereof.

Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium oxide, silicon, manganese, aluminium and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or simethicones. So-called micro- or nanopigments are preferably used in sun protection products. Micronized zinc oxide is preferably used. Other suitable UV filters can be found in P. Finkel's review in SÖFW-Journal 122, 543 (1996) and in Parf. Kosm. 3, 11 (1999).

Besides the two groups of primary sun protection factors mentioned above, secondary sun protection factors of the antioxidant type may also be used. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmole/kg), also (metal) chelators (for example ax-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, Superoxid-Dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Biogenic Agents

In the context of the invention, biogenic agents are, for example, tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts and vitamin complexes.

Deodorants and Germ Inhibitors

Cosmetic deodorants counteract, mask or eliminate body odors. Body odors are formed through the action of skin bacteria on apocrine perspiration which results in the formation of unpleasant-smelling degradation products. Accordingly, deodorants contain active principles which act as germ inhibitors, enzyme inhibitors, odor absorbers or odor maskers.

Germ Inhibitors

Basically, suitable germ inhibitors are any substances which act against gram-positive bacteria such as, for example, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), antibacterial perfumes, thymol, thyme oil, eugenol, clove oil, menthol, mint oil, famesol, phenoxyethanol, glycerol monocaprate, glycerol monocaprylate, glycerol monolaurate (GML), diglycerol monocaprate (DMC), salicylic acid-N-alkylamides such as, for example, salicylic acid-n-octyl amide or salicylic acid-n-decyl amide.

Enzyme Inhibitors

Suitable enzyme inhibitors are, for example, esterase inhibitors. Esterase inhibitors are preferably trialkyl citrates, such as trimethyl citrate, tripropyl citrate, triisopropyl citrate, tributyl citrate and, in particular, triethyl citrate (Hydagen® CAT). Esterase inhibitors inhibit enzyme activity and thus reduce odor formation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

Odor Absorbers

Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. Odor absorbers are not active against bacteria. They contain, for example, a complex zinc salt of ricinoleic acid or special perfumes of largely neutral odor known to the expert as “fixateurs” such as, for example, extracts of ladanum or styrax or certain abietic acid derivatives as their principal component. Odor maskers are perfumes or perfume oils which, besides their odor-masking function, impart their particular perfume note to the deodorants. Suitable perfume oils are, for example, mixtures of natural and synthetic fragrances. Natural fragrances include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, lemon balm oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, p-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Antiperspirants

Antiperspirants reduce perspiration and thus counteract underarm wetness and body odor by influencing the activity of the eccrine sweat glands. Aqueous or water-free antiperspirant formulations typically contain the following ingredients:

astringent active principles,

oil components,

nonionic emulsifiers,

co-emulsifiers,

consistency factors,

auxiliaries in the form of, for example, thickeners or complexing agents and/or

non-aqueous solvents such as, for example, ethanol, propylene glycol and/or glycerol.

Suitable astringent active principles of antiperspirants are, above all, salts of aluminium, zirconium or zinc. Suitable antihydrotic agents of this type are, for example, aluminium chloride, aluminium chlorohydrate, aluminium dichlorohydrate, aluminium sesquichlorohydrate and complex compounds thereof, for example with 1,2-propylene glycol, aluminium hydroxyallantoinate, aluminium chloride tartrate, aluminium zirconium trichlorohydrate, aluminium zirconium tetrachlorohydrate, aluminium zirconium pentachloro-hydrate and complex compounds thereof, for example with amino acids, such as glycine. Oil-soluble and water-soluble auxiliaries typically encountered in antiperspirants may also be present in relatively small amounts. Oil-soluble auxiliaries such as these include, for example,

inflammation-inhibiting, skin-protecting or pleasant-smelling essential oils,

synthetic skin-protecting agents and/or

oil-soluble perfume oils.

Typical water-soluble additives are, for example, preservatives, water-soluble perfumes, pH adjusters, for example buffer mixtures, water-soluble thickeners, for example water-soluble natural or synthetic polymers such as, for example, xanthan gum, hydroxyethyl cellulose, polyvinyl pyrrolidone or high molecular weight polyethylene oxides.

Film Formers

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Swelling Agents

Suitable swelling agents for aqueous phases are montmorillonites, clay minerals, Pemulen and alkyl-modified Carbopol types (Goodrich). Other suitable polymers and swelling agents can be found in R. Lochhead's review in Cosm. Toil. 108, 95 (1993).

Insect Repellents

Suitable insect repellents are N,N-diethyl-m-toluamide, pentane-1,2-diol or Ethyl Butylacetylaminopropionate.

Self-Tanning Agents and Depigmenting Agents

A suitable self-tanning agent is dihydroxyacetone. Suitable tyrosine inhibitors which prevent the formation of melanin and are used in depigmenting agents are, for example, arbutin, ferulic acid, koji acid, coumaric acid and ascorbic acid (vitamin C).

Hydrotropes

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are

glycerol;

alkylene glycols such as, for example, ethylene glycol, diethylene glycol, propylene glycol, butylene glycol, hexylene glycol and polyethylene glycols with an average molecular weight of 100 to 1000 dalton;

technical oligoglycerol mixtures with a degree of self-condensation of 1.5 to 10 such as, for example, technical diglycerol mixtures with a diglycerol content of 40 to 50% by weight;

methylol compounds such as, in particular, trimethylol ethane, trimethylol propane, trimethylol butane, pentaerythritol and dipentaerythritol;

lower alkyl glucosides, particularly those containing 1 to 8 carbon atoms in the alkyl group, for example methyl and butyl glucoside;

sugar alcohols containing 5 to 12 carbon atoms, for example sorbitol or mannitol,

sugars containing 5 to 12 carbon atoms, for example glucose or sucrose;

amino sugars, for example glucamine;

dialcoholamines, such as diethanolamine or 2-aminopropane-1,3-diol.

Preservatives

Suitable preservatives are, for example, phenoxyethanol, formaldehyde solution, parabens, pentanediol or sorbic acid and the silver complexes known under the name of Surfacine® and the other classes of compounds listed in Appendix 6, Parts A and B of the Kosmetikverordnung (“Cosmetics Directive”).

Perfume Oils and Aromas

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat. Suitable aromas are, for example, peppermint oil, spearmint oil, aniseed oil, Japanese anise oil, caraway oil, eucalyptus oil, fennel oil, citrus oil, wintergreen oil, clove oil, menthol and the like.

Dyes

Suitable dyes are any of the substances suitable and approved for cosmetic purposes as listed, for example, in the publication “Kosmetische Färbemiftel” of the Farbstoffkommission der Deutschen Forschungsgemeinschaft, Verlag Chemie, Weinheim, 1984, pages 81 to 106. Examples include cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS(C.I. 69800) and madder lake (C.I. 58000). Luminol may also be present as a luminescent dye. These dyes are normally used in concentrations of 0.001 to 0.1% by weight, based on the mixture as a whole.

The total percentage content of auxiliaries and additives may be from 1 to 50% by weight and is preferably from 5 to 40% by weight, based on the particular preparations. The preparations may be produced by standard hot or cold processes and are preferably produced by the phase inversion temperature method.

### EXAMPLES

Germination. The plant seeds were soaked in water at 14° C. for 5 to 14 h (alternatively: alternately soaked in water and dried in air) and then germinated in special rotating and ventilated culture dishes over a period of 24 to 233 h at 25° C.

Production Example H1. Lentils were germinated in 7 days and then shock-frozen. 440 g of the frozen germs were ground and suspended in 660 ml water. The suspension was stirred for 90 mins. at 20° C. and the insoluble constituents were removed by centrifugation and filtration. The filtrate was then freed from water and freeze-dried.

Production Example H2.500 g of the frozen lentil germs were ground and suspended in 1 liter water. The suspension was stirred for 1 h at 20° C. and the temperature was then increased in steps to 90° C. over a period of 2 h. The solid constituents were then removed again and, after concentration by evaporation, the extracts were freeze-dried.

Production Example H3. Sunflower seeds were germinated in 7 days and then shock-frozen. 500 g of the frozen germs were ground and suspended in 750 ml water. The suspension was stirred for 1 h at 20° C. and then heated for 15 mins. to 100° C. The solid constituents were then removed again and, after concentration by evaporation, the extracts were freeze-dried.

Production Example H4. Sunflower seeds were germinated in 1 day and then shock-frozen. 500 g of the frozen germs were ground and suspended in 750 ml water. The suspension was stirred for 1 h at 20° C. and then heated for 15 mins. to 100° C. The solid constituents were then removed again and, after concentration by evaporation, the extracts were freeze-dried.

Production Example H5. Spelt was germinated in 31 hours and then shock-frozen. 300 g of the frozen germs were ground and suspended in 450 ml water. The suspension was stirred for 60 mins. at 20° C. and the insoluble constituents were removed by centrifugation and filtration. The filtrate was then freed from water and freeze-dried.

Production Example H6.450 g of the frozen spelt germs were ground and suspended in a mixture of 2.5 liters methanol and 0.1 liter water. The germs were then extracted under reflux for 1 hour. After cooling, the methanol was removed in vacuo and the extract was freeze-dried.

Production Example H7. Grains of rye were germinated in 26 hours and then shock-frozen. 200 g of the frozen germs were ground and suspended in 400 ml water. The suspension was stirred for 60 mins. at 20° C. and the insoluble constituents were removed by centrifugation and filtration. The filtrate was then freed from water and freeze-dried.

Production Example H8.450 g frozen rye germs (germinated in 26 hours) were ground and suspended in 2 liters methanol. The germs were then extracted under reflux for 1 hour. After cooling, the methanol was removed in vacuo and the extract was freeze-dried.

Production Example H9.500 g frozen lentil germs (germinated in 26 hours) were ground and suspended in a mixture of 1.4 liters methanol and 0.47 liter distilled water. The germs were then extracted under reflux for 1 hour. After cooling, the methanol was removed in vacuo and the extract was freeze-dried.

Production Example H10. Fresh rye germs were obtained from Germ'Line (France) and frozen. In a reactor, 50 kg of the frozen rye germs were heated to 50° C. with 98 kg water, followed by vigorous stirring for 2 hours. The suspension was then centrifuged and kept at 80° C. for 4 hours. The pH was adjusted to pH 4.5-5 and the suspension was filtered to obtain a liquid extract with a solids content of 13% by weight to which 5% by weight glycerol was then added.

Production Example H11. Fresh lentil germs were obtained from Germ'Line (France) and frozen. In a reactor, 50 kg of the frozen lentil germs were heated to 50° C. with 98 kg water, followed by vigorous stirring for 2 hours. The suspension was then centrifuged and kept at 80° C. for 4 hours. The pH was adjusted to pH 4.5-5 and the suspension was filtered to obtain a liquid extract with a solids content of 2.5% by weight to which 5% by weight glycerol was then added.

Production Example H12. Fresh spelt germs were obtained from Germ'Line (France) and frozen. In a reactor, 50 kg of the frozen spelt germs were heated to 50° C. with 98 kg water, followed by vigorous stirring for 2 hours. The suspension was then centrifuged and kept at 80° C. for 4 hours. The pH was adjusted to pH 4.5-5 and the suspension was filtered to obtain a liquid extract with a solids content of 8% by weight to which 5% by weight glycerol was then added.

Production Example H13. Fresh Kamut® wheat germs were obtained from Germ'Line (France) and frozen. In a reactor, 50 kg of the frozen wheat germs were heated to 50° C. with 98 kg water, followed by vigorous stirring for 2 hours. The suspension was then centrifuged and kept at 80° C. for 4 hours. The pH was adjusted to pH 4.5-5 and the suspension was filtered to obtain a liquid extract with a solids content of 7% by weight to which 5% by weight glycerol was then added.

Production Example 14. The liquid extract of Example H13 was spray dried with dextrin as carrier before addition of the glycerol. A powder containing 50% by weight dry extract of Kamut® wheat germs and 50% by weight dextrin was obtained.

A. Effectiveness Against Ageing of the Skin

The enzyme glucose-6-phosphate dehydrogenase (G6PDH) catalyzes the first step of the “pentose shunt” in which a major constituent of DNA, namely deoxyribose, is formed. In this first step, glucose-6-phosphate (G6P) is converted by G6PDH into 6-phosphatogluconate (6PG). At the same time, the co-enzyme needed for this conversion, NADP, is reduced to NADPH2 which in turn is capable of catalyzing a number of other biological reactions such as, for example, the recycling of glutathione or the synthesis of lipids. Reduced glutathione protects many enzymes having SH groups and cells against oxidative stress, such as UV exposure for example. The G6PDH content is thus an important parameter for cell protection and skin renewal. The G6PDH activity was determined in vitro on human fibroblasts by Okada's enzymatic method; the DNA content was determined by Desaulniers' method. The results are set out in Table 1 which shows the results of three series of measurements involving triple determination in %-rel against a blank.

TABLE 1
Stimulation of G6PDH activity (figures in %-rel)

| | Conc. | DNA | | G6PDH | |
|---|---|---|---|---|---|
| Extract | % w/v | After 3 d | After 6 d | After 3 d | After 6 d |
| Blank | 0.1 | 100 | 100 | 100 | 100 |
| H1 | 0.1 | 152 | 145 | 138 | 100 |
| H3 | 0.1 | 135 | 107 | 181 | 221 |
| H5 | 0.1 | 93 | 89 | 154 | 204 |
| H7 | 0.03 | 74 | 116 | 118 | 103 |

b. Regenerative and Growth-Stimulating Activity

After incubation for 72 h in a nutrient solution, fibroblasts form saturated monolayers, the fibroblasts cease their activity and growth stops. The cell fuel adenosine triphosphate (ATP), which is essentially formed in the mitochondria, is needed to activate certain enzymes which, for example, control the cell skeleton, the ionic channels, the uptake of nutrients and a large number of other important biological processes. The protein content of the cells was determined by Bradford's method [cf. Anal. Biochem. 72, 248-254 (1977)]. Glutathione (GSH) is a special protein which is produced by the cells for protection against oxidative stress and environmental poisons, more particularly against heavy metals. The three amino acids involved in the reduced form of GSH are linked to special cytoplasmatic enzymes which need ATP for activation. An increase in the GSH concentration leads to an increase in the glutatione-S-transferase activity, a detoxifying enzyme. The GHS content was determined by Hissin's method [cf. Anal. Biochem. 4, 214-226 (1977].

The growth-stimulating effect of the test substances was tested on human fibroblasts. In a first series of tests, the fibroblasts were incubated in a nutrient medium for 1 day at 37° C./5% by vol. CO₂, the nutrient medium was replaced by a medium which contained the test substances and the fibroblasts were incubated for another 3 days at 37° C. The protein content of the cells and the ATP concentration were then determined.

The survival-stimulating effect was determined in a second series of tests. To this end, the fibroblasts were incubated first for 3 days at 37° C. in a nutrient solution and then for 3 days at the same temperature in a test solution. The protein content of the cells and the GSH concentration were then determined.

The results are set out in Table 2 which shows the results of 3 series of measurements involving triple determination in %-rel against a blank.

TABLE 2
Growth and survival-stimulating effect (figures in %-rel.)

| | Conc. | Growth test | | Survival test | |
|---|---|---|---|---|---|
| Extract | % w/v | Proteins | ATP | Proteins | GSH/proteins |
| Blank | 0 | 100 | 100 | 100 | 100 |
| H1 | 0.03 | 112 | 101 | 130 | 123 |
| H2 | 0.01 | 100 | 130 | | |
| H2 | 0.03 | | | 111 | 131 |
| H3 | 0.1 | 149 | 135 | 125 | 177 |
| H4 | 0.1 | 129 | 118 | 136 | 105 |
| H5 | 0.1 | | | 126 | 154 |
| H6 | 0.1 | 153 | 182 | 176 | 162 |
| H7 | 0.1 | | | 120 | 146 |
| H8 | 0.1 | 157 | 141 | 144 | 146 |
| H9 | 0.03 | 136 | 169 | 210 | 151 |
| H13 | 3 | 170 | | 166 | 146 |

The Examples show that the extracts of the germs have great potential for stimulating the renewal of skin cells and improving protein synthesis and also for increasing the metabolism in regard to the growth and protection of the fibroblasts.

Accordingly, these extracts are eminently suitable as active components for cosmetic preparations against ageing of the skin or for renewing the structural proteins of the skin, such as collagen, elastin and glycoproteins, and for supporting the healing of wounds. In addition, the extracts according to the invention increase the level of reduced glutathione and thus improve the activity and the protection mechanism of the cells against harmful environmental poisons, such as heavy metals for example, and oxidative stress.

C. Anti-Inflammatory Activity

In the course of cutaneous inflammation, leucocytes, such as the polymorphonuclear neutrophilic granulocytes (PMNs) for example, are stimulated by peptides, such as cytokinins for example, to emit messenger substances, such as leucotriene for example, which are released from activated or necrotic cells in the dermis. These activated PMNs release not only pro-inflammatory cytokinins, leucotrienes and proteases, but also ROS, such as superoxides and hypochlorite anions for example, of which the function is to destroy penetrated pathogenic germs or fungi. This activity of the PMNs during the inflammation is known as so-called respiratory burst and can lead to additional damage in the tissue. To investigate to what extent the test extracts can prevent or reduce the respiratory burst, a cell line of human leukaemic granulocytes of these PMNs was incubated together with the test substances at 37° C. and 5% by vol. CO₂. After the respiratory burst had been initiated by addition of a yeast extract (zymosan) to the cell solution, the release of superoxide anions was determined through their reaction with luminol. The results are set out in Table 3 which shows the cell counts and the quantity of ROS released in %-rel to the standard as the mean value of a series of measurements involving triple determination.

TABLE 3
Anti-inflammatory activity (in %-rel. ± standard deviation)

| | | | ROS |
|---|---|---|---|
| Test product | Conc. (% w/v) | Cell counts | released |
| Blank | 0 | 100 | 100 |
| H1 | 0.1 | 102 ± 4 | 77 ± 17 |
| H3 | 0.1 | 96 ± 6 | 52 ± 18 |
| H4 | 0.1 | 95 ± 9 | 40 ± 37 |
| H5 | 0.1 | 100 ± 7 | 57 ± 14 |
| H6 | 0.1 | 110 ± 5 | 44 ± 35 |
| H8 | 0.1 | 103 ± 4 | 37 ± 12 |
| H9 | 0.1 | 102 ± 5 | 12 ± 3 |
| H13 | 0.1 | 100 ± 6 | 39 ± 24 |

The results show that the extracts have a strong inhibiting influence on the respiratory burst of human granulocytes but do not damage the granulocytes.

E. Protection of Cells Against UVA Radiation

The object of the following in vitro tests was to determine whether the extracts of germinating plants could protect human fibroblasts against oxidative stress and, more particularly, against the effects of UVA rays. UVA was selected as the stress factor because the rays penetrate into the dermis where they lead above all to lipoperoxidation of the cytoplasm membranes. The lipoperoxides formed are split into malonaldialdehydes (MDA) which are responsible for the crosslinking of many biomolecules such as, for example, proteins (enzyme inhibition) or nuclein bases (mutagenesis). To carry out the test, a fibroblast culture was mixed with foetal calf serum and, 2 days later, inoculated with the test substances. After incubation for 36 h at 37° C./5% by vol. CO₂, the nutrient medium was replaced by an electrolyte solution and the fibroblasts were damaged by a predetermined dose of UVA (3-15 J/cm²). After the exposure, the quantity of MDA formed was determined in the supernatant solution by reaction with thiobarbituric acid while the content of proteins in the cell homogenizate was determined by Bradford's method. The results are set out in Table 4 as %-rel against the standard. Table 4 shows the mean value of two series of measurements involving triple determination.

TABLE 4
Activity against UVA rays (figures in %-rel. ± standard deviation)

| | Conc. | | Cellular |
|---|---|---|---|
| Test product | % w/v | MDA released | proteins |
| Control without UVA | | 0 | 100 |
| Control with UVA | | 100 | 101 |
| H6 + UVA | 0.1 | 71 ± 4 | 129 ± 7 |
| H6 + UVA | 0.3 | 57 | 146 |
| H8 + UVA | 0.1 | 40 ± 1 | 147 ± 8 |
| H9 + UVA | 0.03 | 64 ± 3 | 13 ± 3 |

The results show that the extracts have a lastingly positive effect in combatting oxidative stress without damaging the fibroblasts.

E. Protecting Cells Against UVB Radiation

UVB radiation (280 to 320 nm) induces cutaneous inflammation mainly by activating the enzymes phospholipase A2 or PLA2 which release arachidonic acid from the cell walls. The arachidonic acid is converted by cyclooxygenases into prostaglandins which in turn are secreted by the cells. The fixing of prostaglandins of the PGE2 type to special skin receptors leads to reddening and swelling of the skin such as also occurs in cases of sunburn. In cell cultures, the effect of UVB radiation is associated with the release of cytoplasmatic enzymes, more especially lactate dehydrogenase (LDH). To test the anti-UVB activity of the extracts, human keratinocytes were incubated in a nutrient medium (DMEM+FCS) for 3 days at 37° C./5% by vol. CO₂. The nutrient medium was then replaced by an electrolyte solution which contained the test substance and the keratinocytes were damaged by exposure to UVB radiation (50 mJ/cm²). After incubation for another 24 h, the cell count was determined after trypsination and the quantity of LDH released in the supernatant solution was determined by spectrometry. The results are set out in Table 5 which shows the mean value of two series of measurements involving triple determination. The figures represent %-rel to a blank.

TABLE 5
Activity against UVB rays (figures in % rel. ± standard deviation)

| | Conc. | | |
|---|---|---|---|
| Extract | % w/v | No. of keratinocytes | LDH released |
| Blank without UVB | 0 | 100 | 0 |
| Blank with UVB | 0 | 23 ± 5 | 100 ± 0 |
| H1 + UVB | 0.03 | 98 ± 10 | 19 ± 1 |
| H2 + UVB | 0.1 | 39 ± 1 | 52 ± 15 |
| H3 + UVB | 0.1 | 65 ± 2 | 50 ± 1 |
| H5 + UVB | 0.1 | 83 ± 1 | 29 ± 14 |
| H7 + UVB | 0.1 | 104 ± 2 | 24 ± 0 |
| H8 + UVB | 0.1 | 31 ± 5 | 51 ± 2 |
| H8 + UVB | 0.3 | 44 ± 7 | 28 ± 1 |
| H9 + UVB | 0.03 | 41 ± 14 | 16 ± 9 |
| H9 + UVB | 0.1 | 68 ± 15 | 4 ± 2 |
| H13 + UVB | 1 | 66 ± 7 | 18 ± 5 |
| H13 + UVB | 3 | 67 ± 5 | 9 ± 3 |

The results show that the extracts protect human keratinocytes quite considerably against the effect of UVB radiation and hence show anti-inflammatory activity.

F. Immunostimulation

Immunostimulation is the umbrella term for biochemical processes in which messenger substances, such as β-glucans for example, stimulate the body's own defences, for example for binding and secreting toxins and accelerating the renewal of skin cells. It is known that organisms lose this ability with increasing age. Immunostimulation can be observed in vitro on human leucocytes activated beforehand with a yeast extract (zymosan) [cf. Capsoni et al., Int. J. Immunopharm. 10(2), 121-133 (1998)]. A culture of polymorphonuclear neutrophilic granulocytes (PMNs) was incubated with the test substances for 24 h at 37° C./5% by vol. CO₂. The addition of zymosan initiated the respiratory burst. After 30 mins, the PMN count was determined with an automatic cell counter while the quantity of reactive oxygen species (ROS) released in the supernatant liquid was spectroscopically determined with luminol. The results are set out in Table 6 as %-rel against the standard. Table 6 shows the mean value of two series of measurements involving triple determination.

TABLE 6
Immunostimulation (figures in % rel.)

| | Conc. | | |
|---|---|---|---|
| Test product | % w/v | No. of leucocytes | ROS released |
| Blank | 0 | 100 | 100 |
| H1 | 0.01 | 97 ± 3 | 229 ± 39 |
| H3 | 0.001 | 95 ± 5 | 144 ± 29 |
| H5 | 0.001 | 103 ± 4 | 141 ± 44 |
| H7 | 0.001 | 104 ± 4 | 154 ± 34 |

The results show that the test substances stimulate the immune system and lastingly support the body's own defences, more particularly the skin cells.

G. Inhibition of Melanin Synthesis in B16 Melanocytes

Melanin is the pigment responsible for the color of the skin and hair. It is formed in special organelles, the melanosomes, which occur in the melanocytes in the basal layer of the human epidermis. The synthesis of melanin begins with the oxidation of tyrosine to DOPA (dihydroxyphenylalanine) by tyrosinase. DOPA then polymerizes to melanin which is stored in the melanosomes.

To carry out the test, melanocytes (B16 cell line) were incubated for 3 days at 37° C./5% CO₂ in a standard growth medium for cell cultures containing foetal calf serum (FCS). The growth medium was then replaced by a standard medium with which the substances to be tested were adjusted to different concentrations. After incubation for 3 days, the number of living cells was determined through the cell protein content (Bradford's method) and the content of synthesized melanin—recorded in the cell homogenizate at an optical density of 475 nm. The results are expressed in % against a control of pure cell culture medium.

TABLE 7
Results of the inhibition of melanin synthesis in B16 melanocytes

| | Conc. | | Melanin |
|---|---|---|---|
| Test product | % w/v | Cell protein content | content |
| Control | 0% | 100% | 100% |
| Arbutin | 0.5% | 81% | 35% |
| H11 | 1% | 95% | 41% |
| | 3% | 80% | 32% |
| H10 | 3% | 126% | 46% |
| | 10% | 118% | 40% |
| H12 | 3% | 126% | 59% |
| | 10% | 126% | 46% |
| H13 | 3% | 158% | 63% |
| | 10% | 137% | 55% |

The results show that extracts of germinating plants significantly inhibit the synthesis of melanin in B16 melanocytes without any toxic effects on the cells. Accordingly, they may be used as skin whitening agents in cosmetic preparations, more particularly for the treatment of so-called age spots.

H. Protection of Cells Against Heat Shock

The protection of cells against heat shock was investigated in a test on human fibroblasts. To this end, the viability of stressed cells was determined through the content of cellular ATP (adenosine triphosphate). ATP is an energy-rich component which is produced in the mitochondria. Cells need ATP to maintain the enzymes which sustain the cytoskeleton, the ionic channels, the uptake of food constituents and many vital processes.

To carry out the test, human fibroblasts were cultivated in growth medium for 3 days at 37° C./5% CO₂. The growth medium was then replaced by a standard medium with which the substances to be tested were adjusted to different concentrations. After incubation for 2 days at 37° C./5% CO₂, the cells were exposed for 2 hours to a heat shock at 45° C. and then re-incubated for one day at 37° C./5 CO₂.

TABLE 8
Content of cellular ATP after heat shock treatment (mean values -
triple determination of 4 to 5 assays in % against control)

| Cellular ATP content | H10 | H13 | H12 |
|---|---|---|---|
| Control without stress | 100% | 100% | 100% |
| Control with heat shock: | 20% | 18% | 23% |
| 45° C. 120 mins. | | | |
| 45° C. 120 mins. + extract 1% | 30% | 25% | 18% |
| 45° C. 120 mins. + extract 3% | 37% | 42% | 20% |
| 45° C. 120 mins. + extract 10% | 113% | 59% | 42% |

The heat shock left the cells with toxic effects as reflected in a reduction of ca. 80% in the cellular ATP content. The extracts of germinating plants significantly protect the cells or the cell metabolism against heat shock so that the cellular ATP content increases.

The extracts of germinating plants may therefore be used as active substances for protecting cells against oxidative stress, environmental poisons or UV radiation.

I. Detoxifying Activity: Protection of Human Keratinocytes Against Cell-Damaging Gases

The protection of cells against gaseous toxins was demonstrated on human keratinocytes which had been exposed to poisoning by exhaust gases and cigarette smoke.

Human keratinocytes were cultivated for 2 days at 37° C. in a standard growth medium and then adjusted to different concentrations by a standard cell culture medium. They were then exposed to exhaust gases or cigarette smoke for 4 hours at 37° C.

The viability of the cells was measured by an ATP assay through enzymatic luminescence and a reduced MTT assay [Denizot F. and Lang R.: Rapid calorimetric assay for cell growth and survival; J. Immunol. Methods (1986) 89, 271-277].

TABLE 9
Protection against exhaust gases
(mean value of 6 assays in % against control)

| | ATP content | Reduced MTT content |
|---|---|---|
| Control | 100% | 100% |
| Exhaust gases (EG) | 39% | 57% |
| EG + H13 0.3% | 66% | 69% |

The poisoning by exhaust gases produces a distinct reduction in the cellular ATP content and in the level of reduced MTT in human keratinocytes.

The extract of Kamut® wheat germs has a high potential for protecting human keratinocytes against the damaging effect of exhaust gases.

TABLE 9
Protection against cigarette smoke
(mean value of 6 assays in % against control)

| | ATP content | Reduced MTT content |
|---|---|---|
| Control | 100% | 100% |
| Cigarette smoke (CS) | 36% | 71% |
| CS + H13 0.3% | 51% | 81% |

As with the exhaust gases, Kamut® wheat germs have a positive effect in providing protection against cigarette smoke.

J. Activity in Stimulating HSP (Heat Shock Proteins)

Heat shock proteins (HSPs) are specific proteins which are universally synthesized by all cells in response to stress factors, such as excessive temperatures. They are essential to the process of protein configuration. In stressed cells, HSPs are involved in protection and repair mechanisms. They influence unfolded or aggregated polypeptides by conversion back to the active conformation or acceleration of the proteolysis of denatured proteins.

The expression of HSPs is associated with the protection of cells against stress factors. Minimal stress produces an increase in HSPs and, transitionally, improved resistance to further stress.

One of the main heat shock proteins, HSP 72, occurs in the skin and can be detected by immunocytochemistry in keratinocyte cultures. After exposure to stress, HSP 72 is first synthesized in the cytoplasm and, thereafter, can soon be detected in the cell nucleus and a little later in the nucleolus (one of the cell functions of HSP 72 is protecting the nucleolar structure after stress).

To carry out the test, human keratinocytes were cultivated on glass supports in a growth medium containing foetal calf serum. After incubation for 3 days at 37° C./5% CO₂, the cells were treated with extracts of germinating plants and then rapidly heated in an oven to 45° C. over periods of 10, 15 or 20 minutes (heat shock).

After this heat shock treatment, the keratinocytes were incubated for 2 h at 37° C./5% CO₂. To determine HSP, the cells were fixed for 10 minutes in cold methanol and then incubated for one hour at room temperature with monoclonal antibodies against HSP 72 diluted in a ratio of 1:150. They were then washed with phosphate buffer solution (PBS) and incubated for 45 mins. with biotinylated Ziege anti-mouse antibodies diluted in a ratio of 1:50 and then re-exposed for 45 mins. to a streptavidin/fluorescein complex diluted in a ratio of 1:30.

Negative controls were prepared by leaving out the primary antibodies.

After thorough washing with PBS, the immunochemically colored cells were counter-colored for 10 mins. with Evans Blue. The cells were then observed under a Zeiss confocal laser scanning microscope and the immunochemically colored areas were evaluated by image analysis.

The results are expressed as percent of the culture surface occupied by HSP (1st step of HSP location in the cytoplasm) or as the number of colored cell nuclei (2nd step of HSP 72 location in the cell nucleus), based on the total area of the observed field.

TABLE 11
% of surface occupied by HSPs and treatment
with rye germ extract (mean value of 6
determinations ± standard deviation)

| | Assay 1 | | | Assay 2 | | |
|---|---|---|---|---|---|---|
| | Heat shock duration | | | Heat shock duration | | |
| | 0 min. | 15 mins. | 20 mins. | 0 min. | 15 mins. | 20 mins. |
| Control | 0.03 ± 0.1 | 0.97 ± 0.29 | 6.48 ± 1.75 | | | |
| H10 4% (w/v) | 0.01 ± 0.1 | 2 ± 1.39 | 11.11 ± 2.22 | | | |
| H10 11.9% (w/v) | 0.08 ± 0.3 | 1.92 ± 0.56 | 12.21 ± 2.66 | | | |
| H10 19.6% (w/v) | | | | 0 | 3.15 ± 0.39 | 19.86 ± 2.34 |

TABLE 12
Number of cell nuclei colored by HSPs and treatment with rye germ
extract (mean value of 6 determinations ± standard deviation)

| | 0 min. | 15 mins. | 20 mins |
|---|---|---|---|
| | heat shock | heat shock | heat shock |
| Control | 0 | 0 | 5.67 ± 1.37 |
| H10 4% (w/v) | 0 | 0 | 9.83 ± 2.04 |
| H10 11.9% (w/v) | 0 | 0 | 15.50 ± 2.95 |

The results demonstrate that extract of rye germs significantly increases the synthesis of HSPs in keratinocytes after a heat shock.

TABLE 13
Number of cell nuclei colored by HSPs and treatment
with Kamut ® wheat germ extract (mean value
of 6 determinations ± standard deviation)

| | 0 min. heat shock | 20 mins. heat shock |
|---|---|---|
| Control | 0 | 0 |
| H14 0.7% (w/v) | 0 | 12.5 ± 4.46 |

Extract of Kamut® wheat germs also significantly increases the synthesis of HSPs in keratinocytes after a heat shock.

Accordingly, the extracts of the germinating plants improve the defence mechanism of skin cells. The induction of HSPs in the cells accelerates the response to stress factors and improves the defence mechanism against further stress in a preventive manner.

Table 14 shows a number of Formulation Examples.

TABLE 14
Examples for cosmetic preparations
(water, preservative to 100% by weight)

| Composition (INCI) | A | B | C | D | E |
|---|---|---|---|---|---|
| Emulgade ® SE | 5.0 | 5.0 | 4.0 | — | — |
| Glyceryl Stearate (and) Ceteareth 12/20 (and) | | | | | |
| Cetearyl Alcohol (and) Cetyl Palmitate | | | | | |
| Eumulgin ® B1 | — | — | 1.0 | — | — |
| Ceteareth-12 | | | | | |
| Lameform ® TGI | — | — | — | 4.0 | — |
| Polyglyceryl-3 Isostearate | | | | | |
| Dehymuls ® PGPH | — | — | — | — | 4.0 |
| Polyglyceryl-2 Dipolyhydroxystearate | | | | | |
| Monomuls ® 90-O 18 | — | — | — | 2.0 | — |
| Glyceryl Oleate | | | | | |
| Cetiol ® HE | — | — | — | — | 2.0 |
| PEG-7 Glyceryl Cocoate | | | | | |
| Cetiol ® OE | — | — | — | 5.0 | 6.0 |
| Dicaprylyl Ether | | | | | |
| Cetiol ® PGL | — | — | 3.0 | 10.0 | 9.0 |
| Hexyldecanol (and) Hexyldecyl Laurate | | | | | |
| Cetiol ® SN | 3.0 | 3.0 | — | — | — |
| Cetearyl Isononanoate | | | | | |
| Cetiol ® V | 3.0 | 3.0 | — | — | — |
| Decyl Oleate | | | | | |
| Myritol ® 318 | — | — | 3.0 | 5.0 | 5.0 |
| Coco Caprylate Caprate | | | | | |
| Bees Wax | — | — | — | 7.0 | 5.0 |
| Nutrilan ® Elastin E20 | 2.0 | — | — | — | — |
| Hydrolyzed Elastin | | | | | |
| Nutrilan ® I-50 | — | 2.0 | — | — | — |
| Hydrolyzed Collagen | | | | | |
| Gluadin ® AGP | — | — | 0.5 | — | — |
| Hydrolyzed Wheat Gluten | | | | | |
| Gluadin ® WK | — | — | — | 0.5 | 0.5 |
| Sodium Cocoyl Hydrolyzed Wheat Protein | | | | | |
| Extract H1 | 1.0 | 1.0 | 1.0 | — | — |
| Extract H5 | — | — | — | 1.0 | 1.0 |
| Hydagen ® CMF | 1.0 | 1.0 | 1.0 | 1.0 | 1.0 |
| Chitosan | | | | | |
| Magnesium Sulfate Hepta Hydrate | — | — | — | 1.0 | 1.0 |
| Glycerol (86% by wt.) | 3.0 | 3.0 | 5.0 | 5.0 | 3.0 |

(A) Soft cream,
(B, C) Moisturizing emulsion,
(D, E) Night cream

1. Cosmetic preparations containing an effective quantity of an extract of germinating plants.

2. Preparations as claimed in claim 1, characterized in that the plants are selected from the group consisting of alfalfa, bambara nuts, carob, borage, broccoli, buck wheat, Chinese cabbage, peas, peanuts, flax, fennel, cloves, carrots, cress, lentils, corn, melon, parsley, rape, radishes, rice, red cabbage, celery, mustard, sesame, soya, sunflowers, onions and cereals, such as rye, wheat, Kamut® wheat, barley, oats and spelt.

3. Preparations as claimed in claims 1 and/or 2, characterized in that they contain the extracts in quantities of 0.01 to 25% by weight.

4. A process for the production of plant extracts in which the germinating seeds are extracted with water and/or alcohol, the extract obtained is heat-treated and optionally dried, optionally after the addition of exogenous enzymes.

5. The use of extracts of germinating plants for the production of cosmetic preparations.

6. The use of extracts of germinating plants for stimulating cell growth and the cell metabolism.

7. The use of extracts of germinating plants for stimulating the renewal of dermal macromolecules by the fibroblasts.

8. The use of extracts of germinating plants for stimulating cell protein synthesis for protection against spontaneous ageing effects.

9. The use of extracts of germinating plants for increasing the protein and GSH concentrations in the cells.

10. The use of extracts of germinating plants for stimulating G6PDH activity,

11. The use of extracts of germinating plants for immunomodulation.

12. The use of extracts of germinating plants as anti-inflammatory agents.

13. The use of extracts of germinating plants as active components against acne and rosacea.

14. The use of extracts of germinating plants as antioxidants.

15. The use of extracts of germinating plants for protecting the skin and hair against the effects of UVA and UVB radiation.

16. The use of extracts of germinating plants for protecting sensitive skin.

17. The use of extracts of germinating plants an anti-stress agents.

18. The use of extracts of germinating plants for stimulating the synthesis and release of heat shock proteins.

19. The use of extracts of germinating plants as lipolytic agents.

20. The use of extracts of germinating plants as active components for purifying body cells.

21. The use of extracts of germinating plants as active components for inhibiting the synthesis of melanin in skin and hair cells.

22. The use of extracts of germinating plants as active components with oestrogen-like activity.

23. The use of extracts of germinating plants as active components for protection against environmental poisons for detoxifying cells.
